(19) United States
(12) Patent Application Publication
(10) Pub. No.: US 2020/0269538 A1
(43) Pub. Date: Aug. 27, 2020

(54) GLASS AND POLYMER RIGID ARTICLES

(71) Applicant: Bemis Company, Inc., Neenah, WI (US)

(72) Inventors: Ryan A. Michaud, Winneconne, WI (US); Rebecca M. Mick, Appleton, WI (US); Daniel C. Vennerberg, Neenah, WI (US); Christopher L. Osborn, Germantown, WI (US); Edward L. Haedt, Oshkosh, WI (US)

(21) Appl. No.: 16/646,954

(22) PCT Filed: Sep. 12, 2017
(86) PCT No.: PCT/US2017/051060

§ 371 Date: Mar. 12, 2020

(51) Int. Cl. B32B 1/02; B32B 17/06; B32B 27/32; B29C 45/16; C03C 8/24; C03C 8/08; A61M 5/31

(52) U.S. Cl. CPC B32B 1/02; A61M 2207/00; B32B 27/325; B29C 45/16; C03C 8/24; C03C 8/08; A61M 5/3129; B32B 2307/7244; B32B 2307/7246; B32B 2250/03; B32B 2250/40; B32B 2439/70; B32B 2439/80; B32B 2535/00; C03C 2205/00; B32B 17/064

(57) ABSTRACT

Multilayered molded articles comprising a glass layer interposed between first and second non-glass layers are described. These molded articles provide enhanced oxygen and moisture barrier protection to stored contents and are useful in medical, pharmaceutical, food, and research applications as containers and packaging materials.

### FIELD OF THE INVENTION

The invention relates to multilayered three-dimensional molded articles.

### BACKGROUND

There is great interest in the development of high barrier articles which can be used to package, store and transport oxygen and moisture sensitive products. Glass articles, in common use in medical and food storage applications, have good barrier properties, but are prone to breakage. Additionally, glass, although generally non-reactive, is not a completely inert material. Components such as alkali metals that are present in some glass formulations can leach into products on contact. In addition or alternatively, one or more components stored in contact with glass may migrate into the glass. Such migration is referred to as “scalping” of the component by the glass.

These issues have led to the development of polymeric articles, which are both durable and relatively inert. Polymer syringes, for example, particularly cyclic olefin copolymer (COC) syringes, are known to provide a “clean” and inert product contact surface that is more durable than glass. However, polymeric articles lack the barrier properties of glass, and many substances, including many medications, are oxygen- and/or moisture-sensitive and are not adequately protected by polymeric articles.

Layering techniques that generate different polymer layers have been employed to improve the barrier properties of molded polymeric articles. For example, multilayered polymeric molded articles that use ethylene vinyl alcohol (EVOH) as a barrier layer in a two- or three-layered configuration are known. U.S. Patent Application Publication 2014/0120282 describes a multilayered co-injection molded polymeric article formed from a polymeric core layer (EVOH or liquid crystalline polymer) disposed adjacent to a protective layer formed from a base polymer. However, while an EVOH layer may improve an article's oxygen barrier properties, EVOH is hydrophilic and moisture-sensitive. Additionally, moisture absorbance is known to compromise the effectiveness of EVOH as a gas barrier, leading to increased oxygen permeability. See, e.g., Iwanimi et al., Ethylene Vinyl Alcohol Resins for Gas-Barrier Material,” Tappi J 1983 66. 85-90.

### SUMMARY

The invention provides multilayered three-dimensional molded articles that include a glass layer interposed between first and second layers of a non-glass material, such as a polymer. The molded article may be considered a composite structure in that it includes at least three distinct physical layers. The layered construction provides an effective barrier to oxygen and moisture as well as durability, while preventing leaching of glass components into a product in contact with the article. The molded articles are useful for packaging, storing, containing, and transporting oxygen- and/or moisture-sensitive products such as foods, pharmaceutical products, and medical devices.

The use of a glass layer imparts superior barrier properties to the molded articles compared to polymeric molded articles, as the barrier properties of polymers do not match those of glass. Encapsulation or sequestration of the glass layer between two polymer layers may enhance the structural integrity of the molded article and prevents direct contact between the glass layer and a product that is otherwise in contact with the article.

The molded article may itself constitute the finished product, or it may be a sub-unit of a device or container. The molded article has alternating individual layers of glass and non-glass material, and the total number of glass and non-glass layers is open to variation as dictated by the requirements of a particular application. A typical molded article will contain three layers (e.g., one core glass layer interposed between first and second outer polymer layers), but the total number of alternating individual layers of glass and non-glass material may in some embodiments be greater than three.

While the molded articles are primarily described herein with reference to the use of one or more polymers as first and second non-glass layers, it should be understood that the article can incorporate metal or other inorganic material in place of, or in addition to, one or more polymer layers.

As used herein, the terms “comprises”, “comprising”, and grammatical variations thereof are to be taken to specify the presence of stated features: integers, steps or components or groups thereof, but do not preclude the presence or addition of one or more other features, integers, steps, components, or groups thereof.

The multilayered three-dimensional molded articles will be described more fully hereinafter with reference to the accompanying drawings, in which some, but not all embodiments, of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

### BRIEF DESCRIPTION OF THE DRAWINGS

Further features and advantages of the present invention will become apparent from the following detailed description, taken in combination with the appended drawings, in which:

FIG. 1 is a cross section view of an example of a multilayered molded article comprising a core glass layer interposed between first and second non-glass layers;

FIG. 2 is a longitudinal section view illustrating a syringe barrel and

FIG. 3 is a cross section view of the example of the multilayered article of FIG. 1 in use as a container.

### DETAILED DESCRIPTION OF ILLUSTRATIVE EMBODIMENTS

The molded articles of the invention have at least one individual layer of glass (“glass layer”) and at least two individual layers of non-glass material (“first” and “second” non-glass layers), such that the glass is interposed between the first and second non-glass layers so as to sequester or encapsulate the glass within the molded article. As a result, a product in contact with the article will be in contact with the first or second non-glass layer but not the glass layer. In embodiments of the molded article configured as a container, the first layer can be the inner layer of the container, thereby providing an inner surface of the container for contact with a contained item or product, and the second layer can be the outer layer of the container, thereby providing an outer surface for the container. Polymer encapsulation of glass constrains the glass layer and may impart enhanced structural integrity or rigidity to the molded article by holding the finished part together.

The molded article has utility as, for example and without limitation, a syringe, vial, food container, or constituent part thereof. The invention broadly includes a container comprising the molded article, as well as an assembly comprising a container and a product that is stored in the container. The contained product can be a solid, a liquid, or a gas. The term “liquid” is inclusive of a gel, emulsion, dispersion, hydrogel, cream, ointment, paste, and the like. The molded article is particularly well-suited in container applications for products that do not have a fixed shape, such as liquids, including gels and suspensions and gases. Examples of liquids include, without limitation, medicaments, beverages, and chemical solutions. These items all benefit from the superior barrier properties of glass and the durability of a polymer that are advantageously combined in the molded articles of the invention. For example, co-injection of polymer and glass in a co-injection molding process can be used to form a multilayered finished part configured as a syringe barrel. In a pre-filled syringe application, a medicament contacts the chemically inert polymer layer on the inner surface of the barrel, and the polymer layer on the outer surface protects against breakage, while the encapsulated glass layer enhances the barrier properties of the molded article without leaching into the contents of the barrel and/or reducing scalping of the medicament. Pre-filled syringes and vials can be used for the administration of parenteral medication. In addition to its numerous medical applications, rigid glass/polymer co-injection can be extended to food packaging as well, yielding durable, rigid containers that can be used in place of metal cans and glass jars. The molded articles are three-dimensional articles having a fixed shape, as distinguished from flexible, planar (two-dimensional) films, and are well-suited for use as containers and other packaging vehicles in medical, industrial, food, and research applications.

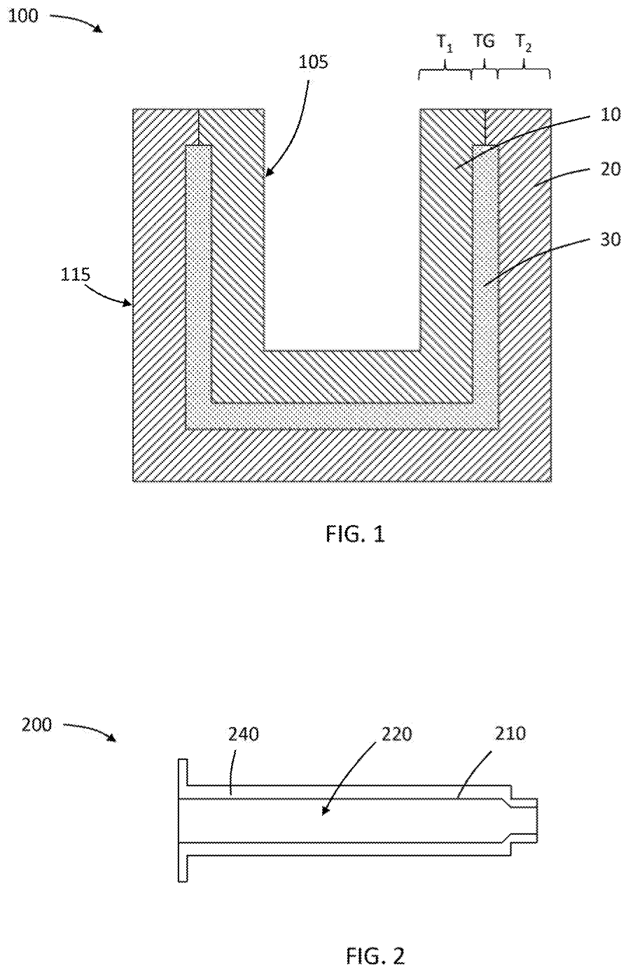

FIG. 1 shows a cross-section of an example of a multilayered molded article configured as a container 100 having inner surface 105 and outer surface 115. Container 100 comprises a core glass layer 30 having a thickness TG interposed between an inner, non-glass first layer 10 having a thickness T₁ and an outer, non-glass second layer 20 having a thickness T₂.

FIG. 2. shows an example of the molded article configured as a syringe barrel 200. Body 240 of syringe barrel 200 comprises the molded article formed from inner and outer non-glass layers and a core glass layer interposed between inner and outer non-glass layers. A liquid present in barrel lumen 220 contacts inner surface 210 of the inner non-glass layer.

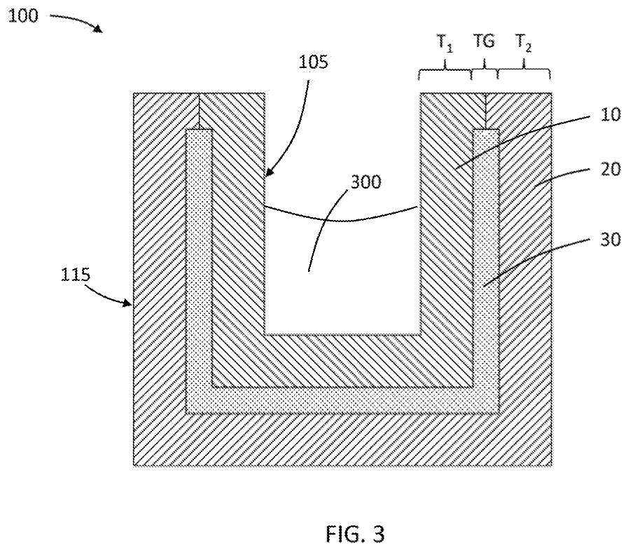

FIG. 3 shows container 100 as in FIG. 1 in use as an assembly to hold a liquid 300. Liquid 300 contained by container 100 is in contact with non-glass first layer 10 but not glass layer 30 or non-glass second layer 20.

The molded articles exhibit superior gas and water barrier characteristics compared to conventional molded packaging materials. In some embodiments, the molded articles have an oxygen transmission rate within a range from 0 to 1 cm³/m²/24 hour at 23° C. and 0% relative humidity. In some embodiments, the molded articles have a water vapor transmission rate within a range from 0 to 1 g/m²/24 hour at 38° C. and 90% relative humidity. Permeation of oxygen and water vapor can be determined using Mocon® permeation-measurement equipment.

Oxygen permeation can be determined at 23° C. and 0% relative humidity, and water vapor permeation at 38° C. and 90% relative humidity. Those skilled in the art will recognize that articles having an oxygen transmission rate within a range from 0 to 1 cm³/m²/24 hour and/or a water vapor transmission rate within a range from 0 to 1 g/m²/24 hour are indicative of defect-free high barrier materials.

The glass (core) layer of the molded article is formed from glass that has a low glass transition temperature (To). For example, the glass of the glass layer of the molded article can have a glass transition temperature, T_{g}, of about 500° C. or less, about 300° C. or less, or about 200° C. or less. For example, the glass transition temperature can be less than 500° C., less than 400° C., less than 350° C., less than 300° C., less than 250° C., less than 200° C., or less than 150° C. In some embodiments, the T_{g} of the glass is less than 350° C. In some embodiments, the glass is an alkali phosphate glass. In some embodiments, the glass is a tin fluorophosphate glass (sometimes referred to as “SnF-glass”). Such glasses can be made by batch melting of inorganic materials such as, but not limited to, BaF₂, SnF₂, ZnF₂, P₂O₅, Sn(PO₄)₂, SnO, Sn₂P₂O₇, SnCl₂, NH₄H₂PO₄, NH₄F, and NH₄PF₆, and can be melted at temperatures not exceeding 600° C. (typically in the range within 400° C. and 500° C.) to provide homogenous glasses of good quality and relatively high chemical durability. Other exemplary glasses include, but are not limited to copper oxide glasses, tin oxide glasses, silicon oxide glasses, tin phosphate glasses, chlorophosphate glasses, chalcogenide glasses, tellurite glasses, borate glasses, bismuth oxide glasses, and combinations thereof. In some embodiments, the glass of the molded article can have a composition comprising, on an elemental basis, tin in a mole percentage of at least 7.4, at least 12.0, or at least 15.4, and at most 17.1 or at most 30.0.

In some embodiments, the glass of the molded article can have a composition comprising, on an elemental basis, fluorine in a mole percentage of at least 4.9, at least 11.2, or at least 19.6, and at most 24.3 or at most 47.2.

In some embodiments, the glass of the molded article can have a composition comprising, on an elemental basis, phosphorus in a mole percentage of at least 6.7, at least 12.1, or at least 14.2, and at most 16.6, at most 19.6, or at most 23.1.

In some embodiments, the glass of the molded article can have a composition comprising, on an elemental basis, oxygen in a mole percentage of at least 20.8, or at least 43.3, and at most 56, at most 61.1, or at most 61.5.

In some embodiments, the glass of the molded article can have a composition comprising, on an elemental basis, tin in a mole percentage within a range of 7.4 to 30, fluorine in a mole percentage within a range from 4.9 to 47.2, phosphorus in a mole percentage within a range from 6.7 to 23.1, and oxygen in a mole percentage within a range from 20.8 to 61.5. In some embodiments, the glass can have a composition comprising, on an elemental basis, tin in a mole percentage within a range from 12 to 17.1, fluorine in a mole percentage within a range from 11.2 to 24.3, phosphorus in a mole percentage within a range from 12.1 to 19.6, and oxygen in a mole percentage within a range from 43.3 to 61.1. In some embodiments, the glass can have a composition comprising, on an elemental basis, tin in a mole percentage within a range from 15.4 to 17.1, fluorine in a mole percentage within a range from 19.6 to 24.3, phosphorus in a mole percentage within a range from 14.2 to 16.6, and oxygen in a mole percentage within a range from 43.3 to 56. In some embodiments, additional elements are present in the glass composition, including, for example, tungsten or niobium.

The qualitative and quantitative determination of the elemental components of the glass compositions of the multilayer molded articles can be determined by energy dispersive x-ray (EDX) spectrometric analysis. EDX spectrometric analysis techniques of inorganic compositions are well-known and can be readily performed by those skilled in the art without undue experimentation.

As noted, the molded article has at least two non-glass layers, such that a glass layer is interposed between first and second non-glass layers. First and second non-glass layers can be composed of the same material or different materials. Any polymer, metal, or inorganic material can be used for a non-glass layer of the molded article. In some embodiments, the first non-glass layer and the second non-glass layer are both polymer layers. As further noted, the molded article is primarily described herein with reference to the use of one or more polymers as the first and second non-glass layers; however, the article can incorporate metal or other inorganic material in place of, or in addition to, one or more polymer layers. In embodiments that incorporate metals or other inorganic materials as the first and/or second non-glass layer, low melting metals and/or low melting inorganic materials are preferred.

In embodiments wherein the first and second non-glass layers are made from identical materials (e.g., polymer A), the structure of the article may have an AB/A-type cross-sectional configuration. First and second polymer layers A, when present on an external surface of the molded article, may be in contact with a product and/or with the external environment. Glass layer B is interposed between two polymer layers A. An illustrative molded article has three layers in an A/B/A configuration wherein A is a polymer and B is a low T_{g} glass.

The thickness of the glass and non-glass layers can be adjusted in accordance with the intended use of the molded article. In some embodiments of the molded article, the thickness of the glass layer can be about 50 micrometers (μm) or less; in some embodiments, the thickness of the glass layer can be about 20 μm or less; in some embodiments, the thickness of the glass layer can be about 10 μm or less. In some embodiments of the molded article, the ratio of the thickness of the glass layer to the thickness of the first layer is 1:3 or less. In some embodiments, the ratio of thickness of the glass layer to the thickness of a second layer is 1:3 or less. For example, the ratio of thickness of the glass layer to the thickness of a first layer can be within a range from about 1:3 to about 1:200, and/or the ratio of thickness of the glass layer to the thickness of the second layer can be within a range from about 1:3 to about 1:200. The glass layer can, for example, be less than 30% of total article thickness (i.e., the combined thickness of all glass and non-glass layers), less than 25% of total article thickness, less than 20% of total article thickness, less than 15% of total article thickness, less than 10% of total article thickness, less than 5% of total article thickness, less than 1% of total article thickness, or less than 0.5% of the total article thickness. The thicknesses of the first and second non-glass layers, T₁ and T₂ respectively, can be the same or substantially the same, or they can be different. For example, when the molded article is used in container applications, an inner layer of non-glass material that is in contact with a contained product may be thicker, or it may be thinner, than an outer layer of non-glass material that is in contact with the external environment.

An example of a polymer for use in a non-glass layer of the molded article is an olefinic polymer, such as a cyclic olefin polymer (COP) or a cyclic olefin copolymer (COC). Examples of commercially available cyclic olefin copolymers include, but are not limited to, the TOPAS® family of resins which is available from Polyplastics (Celanese-Ticona), Tokyo, Japan. Other useful polymers include, without limitation, polypropylene (PP) and polycarbonate (PC), as well as polyolefins such as polyethylenes, ethylene alpha-olefin copolymers, polypropylene copolymers, ethylene vinyl acetate copolymers, ionomers, and blends thereof.

Thermoplastics can be used to form the first and/or second layer of the molded article. A thermoplastic is referred herein as any polymer or polymer mixture that softens when exposed to heat and returns to its original condition when cooled to room temperature. In some embodiments, the polymer may include crystalline or semi-crystalline thermoplastics, amorphous thermoplastics, and blends thereof including, but not limited to aliphatic and aromatic polyamides, polyethers, polyimides, ionomers, aliphatic, and aromatic polyesters such as polyethylene terephthalates, glycol modified polyethylene terephthalates, polyethylene isophthalates, and polyethylene naphthalates, cyclic olefin copolymers, polyolefin homopolymers and copolymers such as polyethylenes, high density polyethylenes, maleic anhydride-modified polyethylenes, ethylene vinyl alcohol copolymers, ethylene vinyl acetate copolymers, ethylene acrylic acid, ethylene methacrylic acid, ethylene alkyl acrylates, and polypropylenes, polyamideimides, polycarbonates, polyetheretherketones, polyetherimides, polyethersulphones, polymethyl methacrylates, polyoxymethylenes, polyphenylene sulphides, polystyrenes including high impact polystyrenes, unplasticized polyvinyl chlorides, thermoplastic polyurethanes, and blends thereof.

In some embodiments, the glass and non-glass material used in the molded articles exhibit similar viscosity-shear rate curves, which facilitates co-extrusion.

In some other embodiments, the glass and non-glass material used in the molded article may exhibit dissimilar viscosity-shear rate curves.

Articles of the invention can be made using any multi-material rigid part process that is capable of forming a multilayered molded article in which a glass layer is interposed between first and second non-glass layers so as to sequester or encapsulate the glass within the molded article. Examples include co-injection molding, insert molding, and over molding. One embodiment of a method for making a multilayered molded article includes heating glass and at least one non-glass material, such as a polymer, introducing the heated glass and non-glass material into a mold to form a glass layer disposed between first and second non-glass layers, and cooling the heated glass and non-glass material to form the multilayered molded article.

In one embodiment, glass and polymer layers are coextruded to form the multilayered molded article. Coextrusion of the polymer and glass layers can be achieved, for example, using multi-component molding processes such as co-injection molding or bi-injection molding. Examples of apparatuses and general methods for multilayer injection molding that can be employed to make the three-dimensional molded article of the invention are found in U.S. Pat. Publ. 2014/0120282 A1 (multilayered co-injection molded article).

In traditional injection molding processes, injection-molded parts are manufactured with a single layer that may be a neat polymer or blend of polymers. In co-injection molding, two materials are introduced into a single mold via separate runner systems. Co-injection molding seeks to create discrete layers which can include neat or blended polymers. An example can be found in some single serve coffee pods, where the co-injected article is a three-layer composite of polypropylene and ethylene vinyl alcohol copolymer (EVOH). The two outermost layers are polypropylene, and the core layer is EVOH.

Using co-injection molding, molded articles can be made by co-injecting glass and a non-glass material, such as a polymer, into a mold to yield a multilayered molded article in which a core glass layer is interposed between first and second non-glass layers. This three-layer configuration effectively sequesters or encapsulates the glass within the molded article such that the glass layer does not constitute a surface of the article. Heated polymer and glass are co-injected into a three-dimensional mold. The polymeric material is introduced into one screw and the glass is introduced into another screw. The molded article can be formed by coextruding layers of glass and polymer together and cooling the extrudate to form a part. The thickness of layers can be adjusted, if desired, such that the first polymer layer is a different thickness from the second polymer layer. Cooling can be accomplished by any convenient method, including but not limited to, subjecting the article to below ambient temperatures, e.g., by refrigeration or fanning, or by allowing the article to cool over time at ambient temperature or at one or a series of pre-set temperatures above ambient temperature.

Co-injection of the glass and non-glass (e.g., polymer) materials can be either simultaneous or sequential. For simultaneous co-injection, an outer (i.e., polymer) material is injected from a first injection unit (usually through a manifold such as those described above) and into a mold cavity. The flow of the outer material into the mold may then be slowed as an inner or core (glass) material from a second source or barrel is injected into the mold, (usually through a co-injection manifold), along with the outer material. In other words, the outer (polymer) and core (glass) mixture may flow concurrently or simultaneously into the mold cavity. This allows the core material to be injected inside the outer material. Subsequently, the outer and core material flow can be terminated substantially simultaneously, or alternatively, the flow of the core material may be stopped while the outer material continues to flow to finish off the part. Alternatively, simultaneous injection may comprise injecting the outer (polymer) material from a first source into the mold cavity, then injecting a core (glass) material into the mold cavity such that core material and outer material simultaneously enter the mold cavity, terminating the flow of the outer material while allowing the core material to continue to flow, terminating the flow of the core material, and resuming and subsequently terminating the flow of the outer material in order to complete the production of a part.

When using sequential co-injection, outer material from a first source is first injected into the manifold to create a flow of outer material into the mold and the mold cavity. The flow of outer material into the mold cavity is then stopped. The outer material may fill approximately 30-50 percent of the mold cavity. Subsequently, the outer material from a second source is used to fill the remainder of the mold cavity and finish the part, or alternatively, the outer material is injected into the mold cavity and toward the very end of the plastic injection, the flow of the outer material may be stopped and the injection of the outer material resumed to provide a better cosmetic appearance to the end product.

After the co-injection of the layers, the article is typically exposed to a “pack and hold” step. During the pack and hold, the pressure is reduced and the temperature is also gradually reduced. As the layers cool, they begin to contract. As a result, the reduced pressure is still maintained and some additional polymer may be introduced into the mold, if desired. After the “pack and hold,” the pressure is further reduced, and the part is cooled while the article remains in the mold cavity. Finally, the mold is opened and the finished article is removed to complete the cycle.

The term “co-injection molding” is meant to encompass co-injection methods whereby two materials from different sources are substantially simultaneously or sequentially injected into a single mold during a single cycle. Co-injection molding, on the other hand, is not meant to refer to forming a part, cooling it, and then layering a material outside the mold over the cooled-part. Co-injection molding is also different from filling one cavity of a two-cavity mold with one material from one barrel and then filling the other cavity with a different material from a second barrel.

It is also not meant to refer to processes that use gas as a core material and then let the gas dissipate to atmosphere-gas assist. Finally, co-injection molding does not include providing a previously-made part and then molding a surface partly or completely thereover. In other words, co-injection molding is different from insert molding or over molding.

### EXAMPLES

### Example 1. Low Temperature Glass/Polymer Co-Injected Article

Co-injection was performed on a model H120RS28122 co-injection molding machine produced by Husky. Cyclic olefin copolymer (COC) available under the tradename TOPAS (TOPAS® family of resins, Polyplastics, Celanese-Ticona, Tokyo, Japan) grade 5013L10 (T_{g} 130° C., melt flow rate 43 dg/min, 1.02 g/cm³) was introduced to a 22 mm 25:1 L/D single screw ram extruder. Tin fluorophosphate glass grade 1648L (T_{g} 130° C.) from Mo-Sci Corporation was introduced to a single screw extruder with a 14 mm screw 25:1 L/D at a 250° C. melt temperature which supplied a piston ram extruder with molten glass.

The two melt streams were fed through a hot runner system heated to 250° C. and simultaneously injected into the mold at a pressure of 78 bar. The melt was cooled in a mold set to 60° C. with an effective cooling time of 8 seconds. The total cycle time of each co-injected part was 13 seconds.

Co-injected parts were cross sectioned and examined with Jeol 6010 SEM with a backscatter electron detector. The contrast was adjusted to highlight the differences in electron density between the two materials to enable the measurement of the glass and total composite thicknesses.

The thickness of the glass layer, T_{glass} or TG, was determined to be about 8 μm, whereas the overall thickness of the co-injected parts, Tₜₒₜₐₗ, was determined to be about 936 μm, for a ratio of T_{glass} to Tₜₒₜₐₗ of about 0.009 (0.9%).

The transmission rate for oxygen was 0.01 cm³/m²/24 hours and the transmission rate for water vapor was 0.01 g/m²/24 hours.

Alternatively, a glass composition can be prepared in house. For example, a batch material of tin fluorophosphate glass can be prepared having a molar composition of 20% SnO+50% SnF₂+30% NH₄H₂PO₄ by melting in a carbon crucible at 500° C. in air in an electric furnace for 15 minutes, casting the molten composition onto aluminum and cooling to room temperature. The cooled sintered glass composition is ground to a particle size of approximately 3 mm.

The above description and examples illustrate certain embodiments of the present invention and are not to be interpreted as limiting. Selection of particular embodiments, combinations thereof, modifications, and adaptations of the various embodiments, conditions, and parameters normally encountered in the art will be apparent to those skilled in the art and are deemed to be within the spirit and scope of the present invention.

1. A multi-layer three-dimensional molded article, comprising:
a first layer;
a second layer; and
a glass layer between the first layer and the second layer, wherein the glass layer comprises glass having a glass transition temperature (T_{g}) of about 500° C. or less.

2. A molded article according to claim 1, wherein the glass comprises an alkali phosphate glass.

3. A molded article according to claim 1, wherein the glass comprises a tin fluorophosphate glass.

4. A molded article according to claim 1, wherein the glass comprises, on an elemental basis, tin in a mole percentage within a range from 7.4 to 30, fluorine in a mole percentage within a range from 4.9 to 47.2, phosphorus in a mole percentage within a range from 6.7 to 23.1, and oxygen in a mole percentage within a range from 20.8 to 61.5.

5. A molded article according to claim 1, wherein the glass comprises, on an elemental basis, tin in a mole percentage within a range from 12 to 17.1, fluorine in a mole percentage within a range from 11.2 to 24.3, phosphorus in a mole percentage within a range from 12.1 to 19.6, and oxygen in a mole percentage within a range from 43.3 to 61.1.

6. A molded article according to claim 1, wherein the glass comprises, on an elemental basis, tin in a mole percentage within a range from 15.4 to 17.1, fluorine in a mole percentage within a range from 19.6 to 24.3, phosphorus in a mole percentage within a range from 14.2 to 16.6, and oxygen in a mole percentage within a range from 43.3 to 56.

7. A molded article according to claim 4, wherein the glass has a T_{g} of about 300° C. or less.

8. A molded article according to claim 5, wherein the glass has a T_{g} of about 200° C. or less.

9. A molded article according to claim 1, wherein the first layer comprises a polymer and the second layer comprises a polymer.

10. A molded article according to claim 9, wherein the first and second layers comprise the same polymer.

11. A molded article according to claim 10, wherein the first and second layers comprise a cyclic olefin copolymer (COC).

12. A molded article according to claim 1, wherein a ratio of thickness of the glass layer to a thickness of the first layer is 1:3 or less, and wherein a ratio of thickness of the glass layer to a thickness of the second layer is 1:3 or less.

13. A molded article according to claim 4, wherein the ratio of thickness of the glass layer to the thickness of the first layer is in a range from about 1:3 to about 1:200, wherein the ratio of thickness of the glass layer to the thickness of the second layer is in a range from about 1:3 to about 1:200.

14. A molded article according to claim 1, wherein the glass layer has a thickness of about 50 micrometers or less.

15. A molded article according to claim 4, wherein the glass layer has a thickness of about 20 micrometers or less.

16. A molded article according to claim 1, wherein thicknesses of the first and second layers are substantially the same.

17. A container comprising the molded article according to claim 1.

18. An assembly comprising:
the container of claim 17; and
a medicament stored in the container.

19. An assembly according to claim 18, wherein the container forms at least a part of a syringe.

20. A method for forming a molded article according to claim 1, comprising:
heating the glass and at least one non-glass material;
introducing the heated glass and non-glass material in a mold to form a glass layer disposed between first and second non-glass layers; and
cooling the heated glass and non-glass material to form the article.
